(19) United States
(12) Patent Application Publication
(10) Pub. No.: US 2025/0165928 A1
(43) Pub. Date: May 22, 2025

(54) SYSTEM AND METHOD FOR DISABILITY ASSESSMENT AND MANAGEMENT

(71) Applicant: ASPYR DIGITAL INC., Toronto (CA)

(72) Inventor: Yvonne CHAN-NICHOLSON, Toronto (CA)

(73) Assignee: ASPYR DIGITAL INC., Toronto, ON (CA)

(21) Appl. No.: 18/833,010

(22) PCT Filed: Jan. 25, 2023
(86) PCT No.: PCT/CA2023/050090

§ 371 Date: Jul. 25, 2024

Related U.S. Application Data
(60) Provisional application No. 63/302,708, filed on Jan. 25, 2022.

(51) Int. Cl. G06Q 10/1057

(52) U.S. Cl. CPC G06Q 10/1057

(57) ABSTRACT

A disability management tool is provided. The disability management system allows for efficient data collection and analysis of claim related data for consistent claims adjudication and claims management by reducing user biases. The data is processed against stored profiles and business rules to reliably determine actions and improve expected outcomes. Aggregated data provides predictive analytics and Return on Investment calculations.

### CROSS REFERENCE TO RELATED APPLICATION

The present application is a non-provisional application of U.S. Provisional patent application No. 63/302,708 filed Jan. 25, 2022 the entirety is incorporated by reference for all purposes.

### TECHNICAL FIELD

The present disclosure relates to disability management and in particular to electronic claims adjudication and managing disability claims.

### BACKGROUND

When a worker/employee becomes ill or injured and can not perform the duties of a job, disability procedures can be utilized to adjudicate claims which provide a means to compensate the worker. To return an ill or injured worker back to work, ongoing claims management to provide assessment, treatment and occupational health and safety services enable the worker to recover with a shorter disability duration and rejoin the workforce. Existing disability management processes are manual, paper-based, processes without consistency, analytics or management overview. These processes are prone to variability and biases in terms of claims adjudication, treatment plans, duration and costs resulting in inefficiencies and inconsistent results. Further, the collection of assessment data across cultural and language groups can present challenges in how information is presented to a patient and collected and conveyed to assessing parties against defined assessment metrics. Accordingly, systems and methods that enable improve disability management software remains highly desirable.

### BRIEF DESCRIPTION OF THE DRAWINGS

Further features and advantages of the present disclosure will become apparent from the following detailed description, taken in combination with the appended drawings, in which:

FIG. 1 shows a representation of system for disability management;

FIG. 2 shows a method of disability management collection;

FIG. 3 shows a method of disability management evaluation;

FIG. 4 shows a process of disability management;

FIGS. 5 & 6 show the employee/client data collection interfaces;

FIG. 7 show a numeric scale for pain scale data collection input;

FIG. 8 shows the collection of data from a manager regarding the employee injury;

FIGS. 9 & 10 show identification of the location of the injury on the employee;

FIG. 11 shows collection of accident details regarding an employee from a manager;

FIG. 12 show an interface of employee satisfaction ratings;

FIG. 13 shows an interface of work injury trends;

FIG. 14 show an interface of a disability dashboard at a corporate level; and

FIG. 15 show a method of adapting to language/cultural requirements of employees in the disability management process.

It will be noted that throughout the appended drawings, like features are identified by like reference numerals.

### DETAILED DESCRIPTION

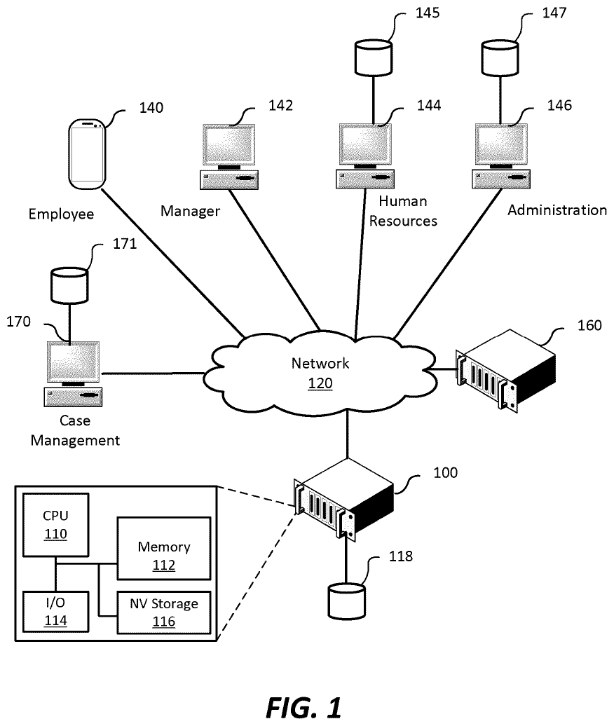

Embodiments are described below, by way of example only, with reference to FIGS. 1-15.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method of disability management. The method also includes receiving at a management server identification of evaluation requirements for a disability condition associated with an employee of an associated case identifier; generating a collection profile for the employee, determining a cultural profile associated with the employee, determine data collection requirements for associated disability condition, converting data collection requirements based on cultural profile associated with the employee, presenting a data collection interface to employee through a remote computing device, receiving disability assessment responses through the data collection interface from the remote computing device, converting received disability assessment responses to standard format from a determined translation mapping based upon the associated cultural profile, processing collected data against a plurality of stored profiles, generating a disability compensation profile, determining expected outcomes associated with the disability compensation profile, processing events associated with the disability compensation profile, and generating one or more analytics from the processed events. Other embodiments of this aspect include corresponding computer systems, apparatus, and computer programs recorded on one or more computer storage devices, each configured to perform the actions of the methods.

Implementations may include one or more of the following features. The method where the data collection requirements are associated with a base collection template, the base collection template defining one of more measures associated with the determined disability compensation profile. An employee language or cultural requirements is selected from a plurality of language or cultural definitions. The employee language or cultural requirements is defined by one or more of a language mapping, a condition expression terminology, rating scales, or graphic mappings. The method may include determining if the generated compensation profile matches and existing profile. The method may include generating a new profile category if an existing profile does not exist. The method may include generating profile tasks and threshold based upon the existing profile. The method may include determining an expected outcome for the disability compensation profile. The method may include generating a recommended approval or denial for the case identifier is generated and presented for selection. The method may include generating profile events when the case identifier is approved defining actions to be performed in relation to the employee claim process. The method may include comparing collected case events with the determined existing profile and identifying when one or more thresholds are exceeded for the case identifier. The method may include aggregating case identifiers into an aggregate event associated with a disability compensation profile. Implementations of the described techniques may include hardware, a method or process, or computer software on a computer-accessible medium.

The present system and method provides for disability management to assist claims adjudication and claims management in order to assist with the return to work and accommodation process, as required by federal law. The system utilizes computing resources for data collection, creating a care plan and performance management.

The present disclosure relates to healthcare algorithms in the area of disability management to assist claims adjudication and claims management in order to assist with the return to work and accommodation process, as required by federal law. The system and method described provide a self-start absence management using an application designed to facilitate the front process of claims adjudication and eventual accommodation in the workplace. The system allows employees to objectively communicate issues with employers and healthcare professionals, while eliminating conscious or unconscious bias by using an internationalization feature to modify text in order to account for an employee's ethnic origin, language, cultural values and place of residence. The conversion of data collection based upon an employees requirement helps address healthcare practitioner's conscious or subconscious bias toward an employee by eliminating personal attitudes/beliefs through standardization of data to adjudicate disability;

The system and method facilitate the improvement of the well being of employees and the impact on their health. The goal of system assimilates data from assessment, evaluation, case management and claims reporting using defined health assessment algorithms and accepts multiple health data sets and flattens this data for use and analysis. This flattened data can be used to fill out necessary health forms and for aggregated data analytics.

The system 100 of FIG. 1 contains modules for each person in the disability management process, for example employee, manager, human resources, administration and case management. Each module collects different data, to gain different disability perspectives as well as to help with completion of forms or initiation of formal processes including claims adjudication. Data collection is provided through a client interface to an employee 140 allowing a person to objectively describe their disability, in various languages, so that the claim can be adjudicated and ongoing claims management can occur in order to optimize their healthcare journey to recovery. The user is able to toggle between languages available (including but not limited to English, French, Spanish) and identify cultural, ethic, geographic differences which may impact how the data is collected. The interface provides specific questions designed to collect key data points and provide objective metrics to determine support or non-support of a disability claim and ongoing disability management. The data interfaces are also provided for manager 142, human resources 144, administration 146 and case management 170 to collect objective data points and provide analysis results. The system 100 communicates with clients or data repositories through a network 120 for receiving and providing data. Data repositories for HR data 145, administrative data 147 and case management data 171 may be retrieved remotely for associated computing systems. The software leverages a multi-language support feature (internationalization) to support multiple modules. This multi-language support feature enables existing graphic icons to be changed to allow for cultural appropriateness. A web service 160 is provided for managing an open application programming interface or retrieving and normalizing data sources. The system 100 stores collected data 118 for evaluation and analytical processing. The system 100 can comprise one of more computing devices in a local, distributed or cloud based computing environment. At least one computing device comprises a CPU 110, memory 112, input/output interface 114 and non-volatile storage 116 for storing instructions providing the disability management software executed by the processor 110. The data collected is provided through a guided data collection process having defined data points used to evaluate conditions related to a disability claim. The modules interacting with respective client computing devices tailor the data collection and presentation elements available to the end user. The system 100 also provide translation mapping to provide internationalization of the data collection system. The internationalization allows for the data collection to be tailored to accommodate language, ethnic and cultural differences, rating scales, and graphical presentations unique to each employee and normalize the data such that the adjudication is performed subjectively minimizing possible biasing conditions. The system may be integration with electronic medical record system, employee management systems or enterprise resource management to retrieve patient related data and/or provide updates in regards to changes in an employee's status.

Real-time dashboards on performance, costs, and savings allows for real-time reporting of Return on Investment are provided through analysis of collected case event data. The case event data is processed using machine learning and artificial intelligence to identify trends and thresholds to aid in claims adjudication, case management and reporting. Proprietary health algorithms enable culturally appropriate intake, triage, claims adjudication, case management and re-assessment. Managers and users can easily navigate, review data, report, and invoice.

After user login, user has option to “create new case”. User can also go to main menu at any time to “create new case”. After manager login, the manager has option to “create new case”. The manager can also go to main menu at any time to “create new case”. After a case has been created, the user/manager can attach files directly to the case file. Documents can be in various formats including pdf, doc, or pictures. Users/managers can record employee human resources (HR) profile records in cases and can manage cases even when employee records are not in the system. Any new cases created will show up in the user/manager home page. After the users/manager records the employee's HR profile, either by creating a new case or by upload, the software will auto populate HR profile information in online submissions forms and in case files. The user/manager can edit the auto populated data fields.

After a case has been created, the user/manager can review and edit all fields in a case file by pressing the “edit” button. After a case has been created, the software will provide a notification to authorized users (including the employee) that a new service request has been submitted. This will prompt those who have a next step in the work flow to complete the next step. After login, the user/manager can check the status of a case by selecting “Go to Cases”. User/manager can also select if they wish to see “Pending Cases” or “Completed Cases” or “All Cases” from the main menu. The manager will receive a text message to notify them about the status of a case. After a claim is adjudicated, the system will generate a status letter for the manager and employee to view. There are pre-defined business rules and work flows for the user, manager and employee. Each will receive a text or email prompt advising them to do the next step in the work flow. At the Company Set Up page, there is an option to add in an “External Contact”. If this is entered, this external contact can get notifications by text or email regarding the status of a case.

The system allows the user/manager to attach files directly to the case file. Documents can be in various formats including pdf, doc, or pictures. The system allows case communication to be recorded by the user/manager. The system allows the user/manager to create notes and supporting documents for a particular case file which will create a report in an adobe readable format. In addition, users can upload notes and supporting documents to particular case files created in PDF, JPEG, or Microsoft Word. After a case has been created, the user/manager can see all of the attached files and reports associated with each case file. Documents can be in various formats including pdf, .doc, or JPEG. The system provides generic and customizable templates, with prompts created from best practice industry standards for what types of information should be collected. Standardized reports can be generated by the system. A search function can accept keywords including employee, case/claim number. For each case file, the gear button at the top right hand corner provides an option to print documents directly from the case file. The essence of time is key to the business rules behind the software. The software is able to track time and base processes on time stamps. The system automatically updates checklists as data is entered or the outstanding task is completed. The system provides different types of keying functionalities. The system distinguishes between mandatory data and optional data.

The system distinguishes between data which should not be deleted or altered by a user type and data which can be edited by a user type. The system allows a user to “save and continue” at each step of the process. The system provides a satisfaction survey at the end of the questionnaire process. This data is then aggregated, and the employee or manager satisfaction rating can be shown by groups, for example by region, department, shift. If a person changes positions, the records will continue to be tracked and related. The system verifies and tracks the reason for the injury or illness, to ensure that the appropriate insurance benefit has been applied. If an employee transitions between a work injury and a non-work injury, the system is able to track that as two separate but related cases. If a case is transitioned to health re-assignment, this can be tracked and the case be related to the same employee. The system tracks and relates all prior closed or other active claims to the same employee. The system allows case communication to be recorded by the user/manager.

The system allows a case to be assigned (routed) to other authorized users. The system allows authorized users to view and monitor their work items, including providing them with due dates and status of each file. The system provides a dashboard which calculates duration of absence between first day of absence and other events, including “Full Return to Work” and “Partial Return to Work”. The formula to calculate dates and days are customizable (e.g., include weekends, do not include weekends). The system can be configured by each individual user to meet their specific needs and preferences, such as language and night mode. The system helps to complete the required forms. Once these forms are completed, they are stored in the case file of the employee, and the authorized user can search and select the forms they wish to retrieve.

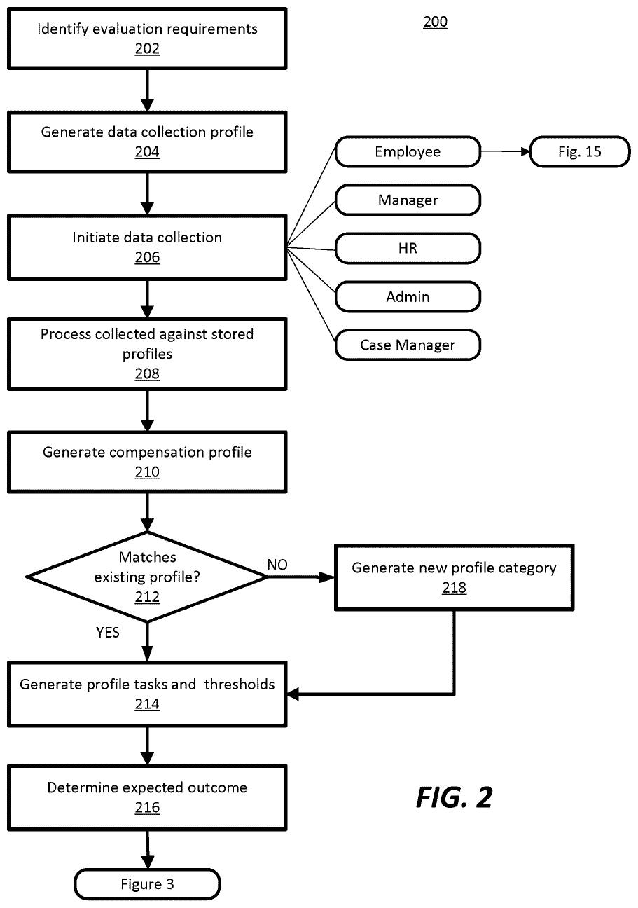

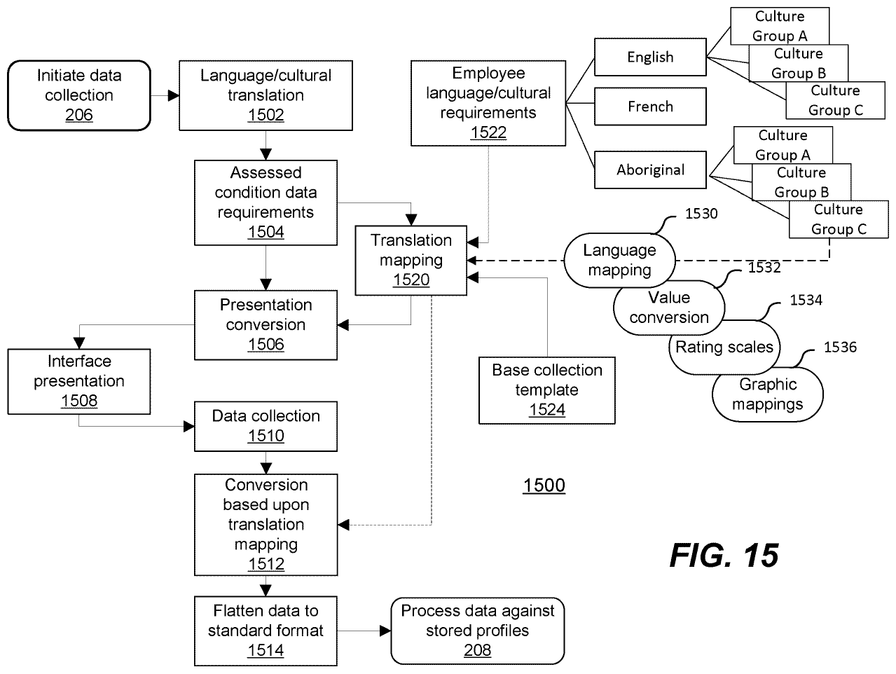

FIG. 2 shows a method 200 of disability management collection. Evaluation requirements are identified (202) associated with the organization, workplace or employee job. A data collection profile is generated (204) defining what data needs to be collected and from which people or systems. Data collection is initiated through presentation of web interface for accessing data repositories. The interaction is performed with the employee and manager to collect subjective and objective data associated with the injury. The employee will be presented a collection interface that can be provided through a mobile application on a mobile device or via a desktop computer if required. Data is collected or retrieved from human resources and administrative sources. At this point the preference of the employee as to how the data is collected can be utilized to modify the data collection process (FIG. 15) based upon language, cultural, ethnicity, or geographic requirements. The collected data is processed against stored profiles (208) stored in the system associated with aspects associated with the claims such as but not limited to job type, employee demographics, and geographic data. A compensation profile (210) can then be generated which can include treatment and expected costs for the resolution of the injury and return to work. If the generated profile does not match existing profiles (No at 212) a new profile is generated (218). If there is a match with an existing profile (Yes at 212) profile, task and thresholds are generated (214). An expected outcome can then be determined (216) in relation to the claim.

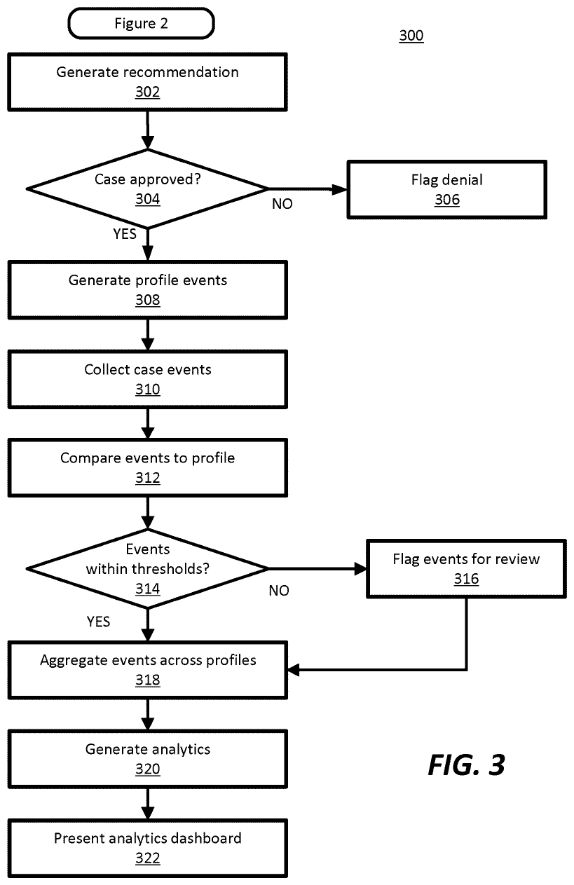

FIG. 3 shows a method of disability management evaluation. Based on the data collected, the claim can be adjudicated. A recommendation is generated (302) based upon the disability condition data collected to identify if it is a valid condition and a treatment profile can be created. If a case manager does not support the claim (No at 304), the claim is flagged for denial (306). If the case manager approves the claim (Yes at 304) events for the profiles (308) can be generated to define data collection (310) in regards to progress and expenses related to the claim. The events can define services that are provided to the employee toward rehabilitation, follow-up data collection, medical services, counselling services, workplace assessment or accommodations, and re-assessment milestones in addition to cost and time metrics. Events can be compared to the profile (312) to determine if they are within the determined threshold such as cost or time. If the events are not within the determined threshold (No at 316) they can be flagged for review. For example, if exceptions in the treatment plan, nature of the injury, or cost profile changes, the exceptions can be identified for consideration and defined as exceptions or for inclusion to the base profile. If the events are within threshold (Yes at 314), they can be aggregated across profiles (318) to generate or define events and thresholds and refine population metrics. For example the average time of work and costs for a particular injury can be determined. Analytics are generated (320) and presented in an analytics dashboard (322). The analytics can be aggregated for a company, across industries or particular work environments to help assess the impact of the disability process.

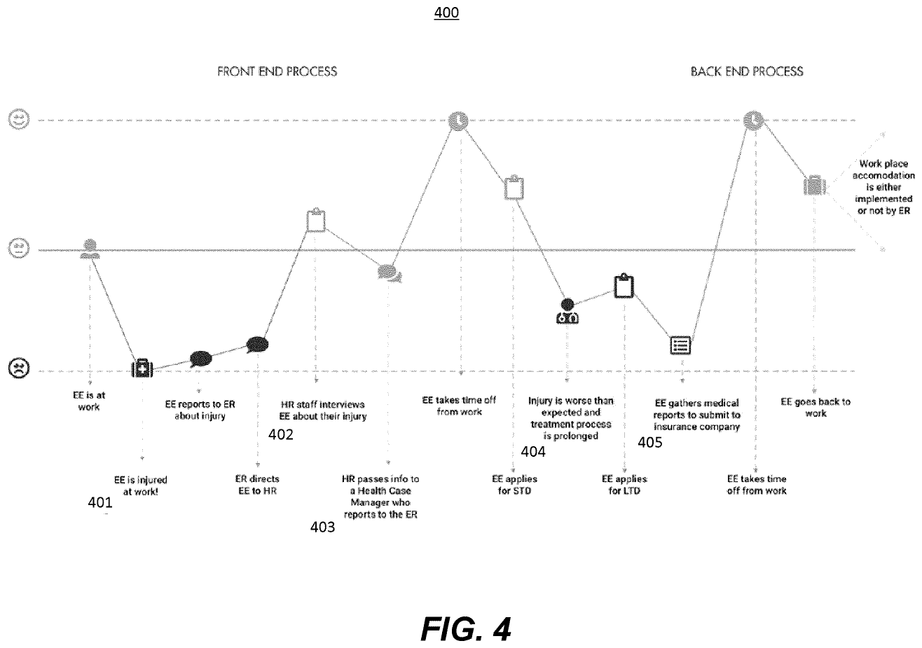

FIG. 4 shows a general process of disability management. The process 400 show the processes from when an injury occurs, the internal HR processes, initiation of health case management, disability claims and return to work and/or accommodation. When an employee is injured at work (401) the employee reports the injury to an administrator start a disability process which is directed to HR. The HR staff collects initial information about the injury (402) and passes the information to a health case manager (403). The employee can then start the disability process (404) to proceed with treatment. The employee must gather medical reports (405) and submit them to the insurance company for processing with the hopeful resolution of the employee returning to the work place with or without accommodation. The existing workflow provides limited data collection to determine a treatment plan and manage outcomes of the process and does not provide any prediction as to time and costs outcome with any level of accuracy. In addition the employee information is provided through traditional standard data collection not tailored to specific injury metrics that can help identify outcome time and costs. Further the determination of the expected outcome is limited by how data is collected from the employee in not accounting for cultural, ethnic, language or geographic differences which can have an impact on the quality of the data collection.

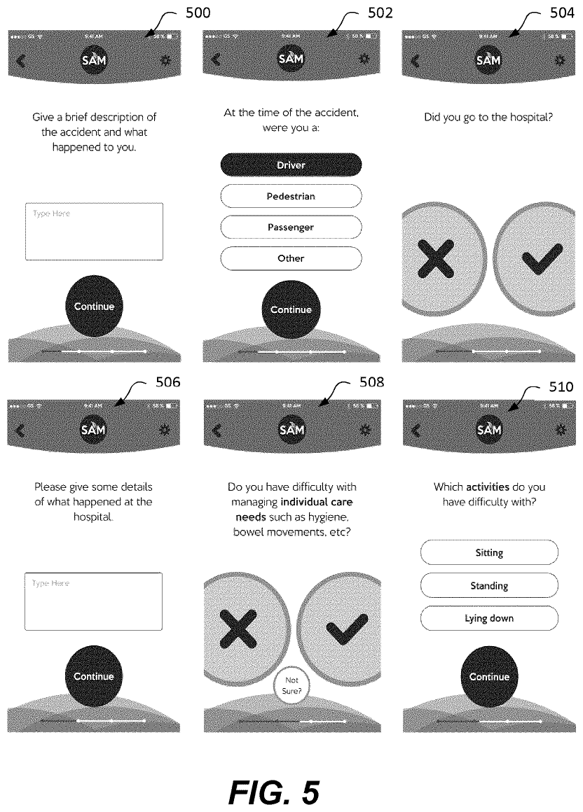

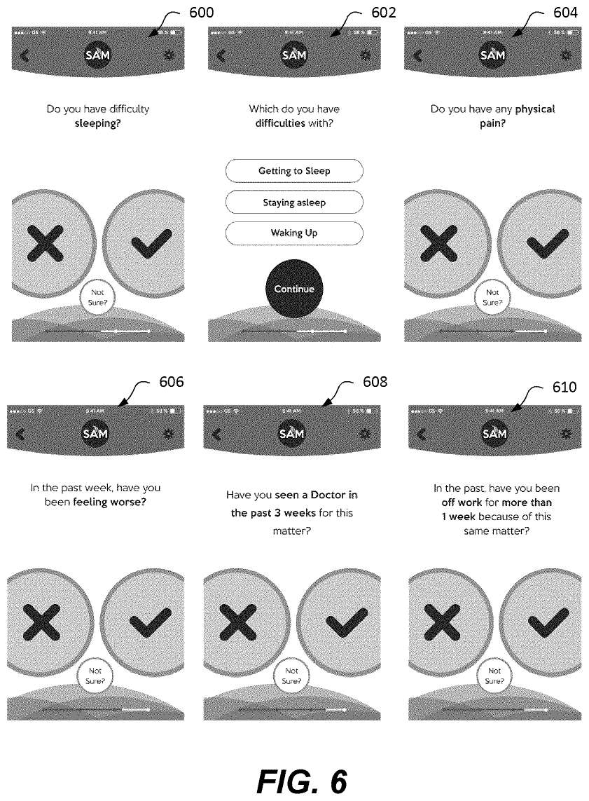

FIGS. 5 and 6 present employee/client interfaces 500-510 and interfaces 600-610 for collection of information regarding the injury and resulting issues from the employee to improve the disability management process. The questions are derived to provide defined metric assessment for collection of data in order to aid in determining outcomes from an injury and possible costs implications during the disability period. Choices for each aspect are provided to the user relating the defined metrics which will provide insight in deriving metrics associated with expected outcomes and treatment events. For example in 504 the need to visit the hospital for an injury may define the severity of an injury and may also identify that additional data is available in terms of assessing the injury. Likewise 510 can be utilized to determine potential accommodation requirements for the employee to return to work or the impact on their current job duties. The progress of the employee can also be tracked 604 and identify if progress is being made 608 and 610 to improving the condition and expected time to conclude a claim process.

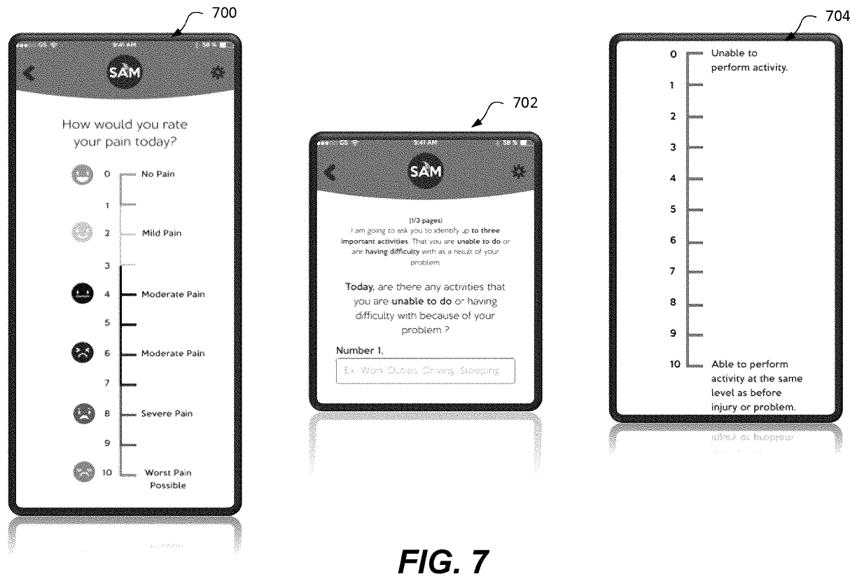

As shown in FIG. 7, a numeric scale may be provided for collection input. For example, interface 700 and 704 provide metric input from the user where interface 702 enables collection of evidence based outcome measures such as using a Visual Analogue Scale or Patient Specific Functional Scale. The scale can be used to determine severity of an injury or an aspect of an injury and determine if improvement has occurred in the treatment process. The metric define by the scale may be determined through alternative presentation of user input and normalized against a scale. For example an intensity slide, or graphic selection may be utilized.

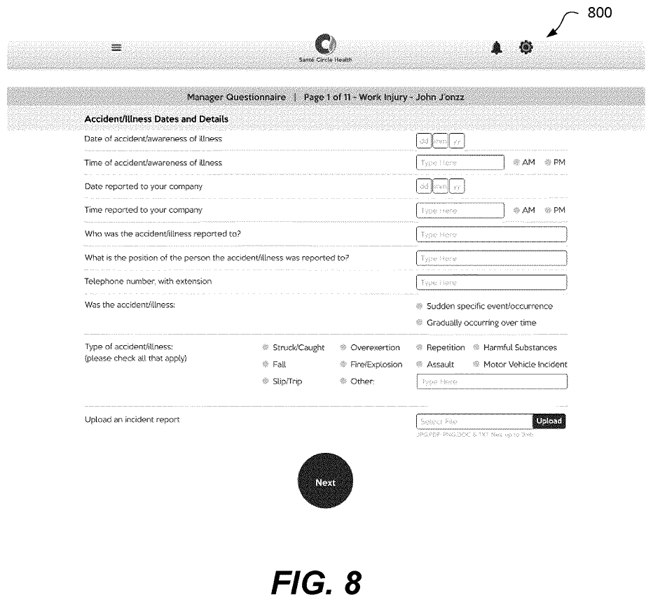

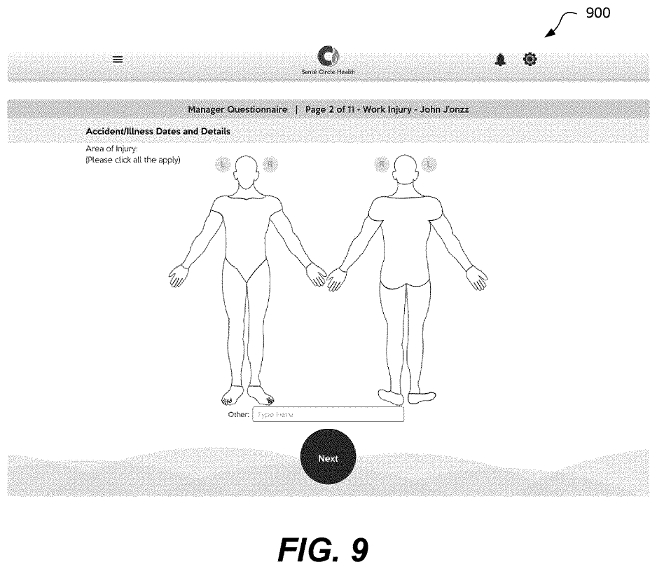

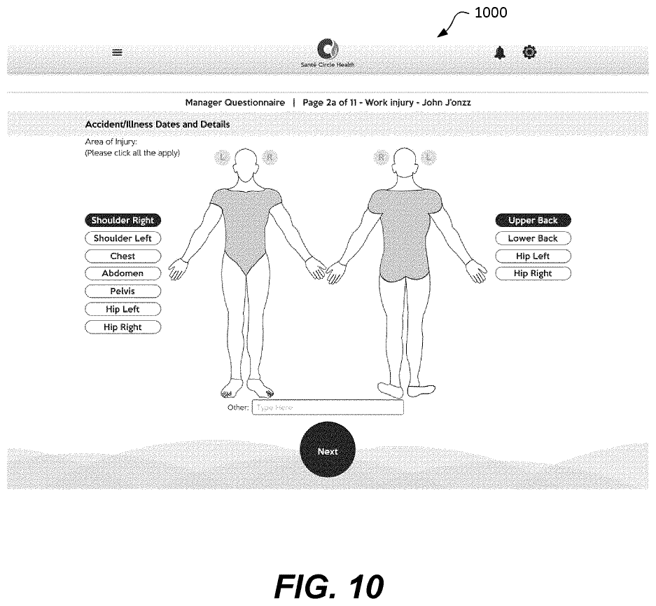

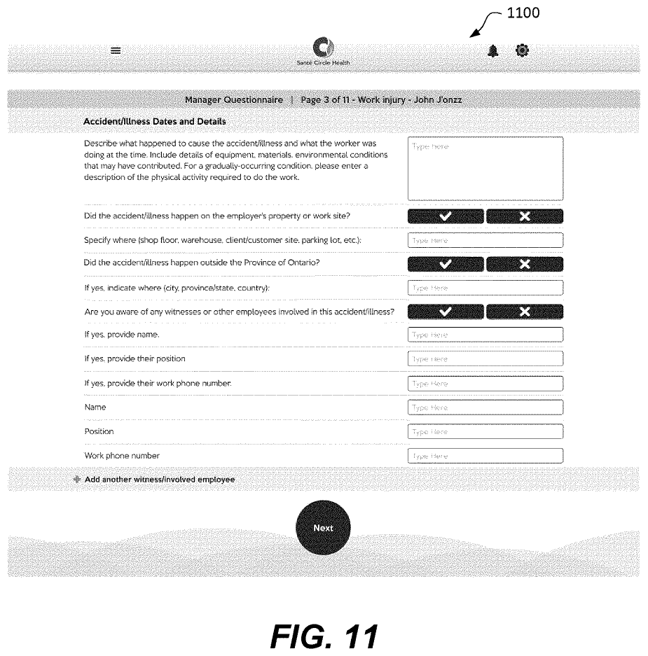

FIGS. 8-11 show interfaces 800, 900, 1000, 1100 presented to a manager for collection of injury information. FIG. 8 shows the collection of information regarding the work injury of the employee related to the initial event causing the work related injury. FIGS. 9 and 10 enable identification of the location of the injury on the employee for the assessment process in a graphic means to help an adjudicator to assess severity and impact on the works tasks required of the employee. FIG. 11 shows collection of accident dates and details from the manager in regards to the accident/illness of the employee which can be used to verify the information provided by the employee.

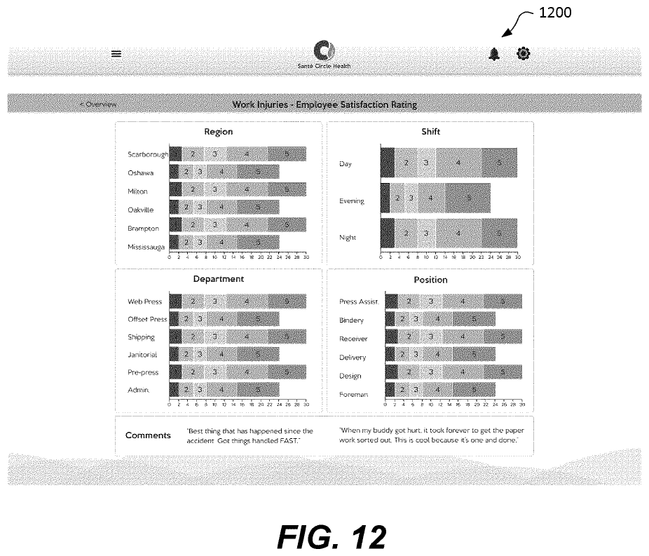

FIG. 12 shows an interface 1200 presenting employee satisfaction based upon the data collected in regards to claims and the evaluation and rehabilitation process. The satisfaction can be identified based upon corporation relevant factors such as region, shift department or position. The satisfaction may also be identified across industry or peer corporations.

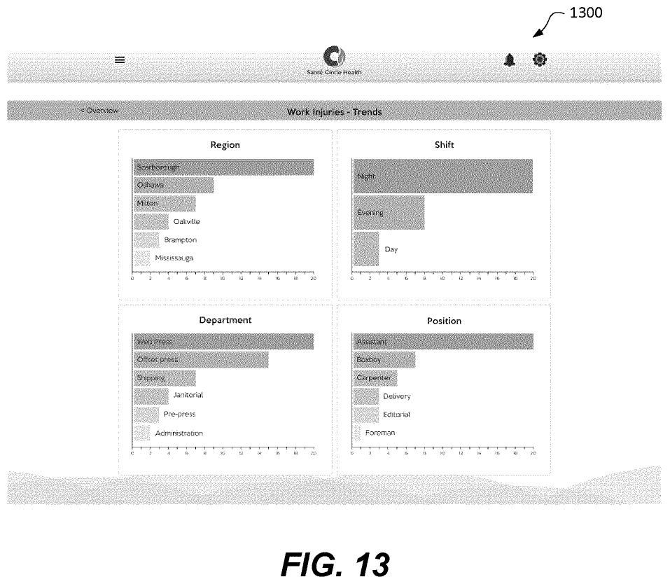

FIG. 13 shows an interface 1300 presenting work injuries trends by location, shift, department and position to identify possible trends in regards to occurrence of injuries in the workplace. The trends can help identify areas of increased risk which may result in increased disability claims.

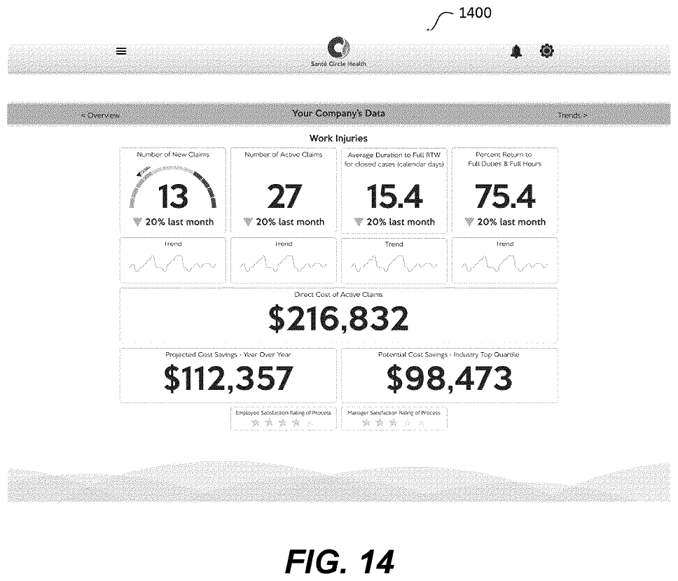

FIG. 14 show an interface 1400 presenting a disability dashboard at a corporate level identifying financial performance in regards to the claims that have been made and the costs per claim, duration of claims and trends. The dashboard can help identify improvement in workplace performance and the risk of injury as well as the costs incurred.

FIG. 15 shows a method 1500 of adapting to language/cultural requirements of employees in the disability management process. During the collection process as shown in FIG. 2 (206) language/cultural translation can be performed (1502) of the employee interface during the collection and assessment process. Based upon the condition/injury that has been identified the data requirements are determined (1504). A translation mapping (1520) is retrieved based upon employee language/cultural requirements (1522) by providing an internationalization ability to convert data collection to meet the needs of an employee facilitating better expression of issues and needs. For example if the employee is aboriginal they may be associated with a particular cultural group and wish to have the assessment interface presented in a manner more appropriate or more easily understand in terms of language, presentation or assessment criteria. Each culture group may have mapping modules such as for example, language mappings 1530, values conversion 1532, rating scales 1534 and graphic mappings 1536. The cultural groups can be based upon an employee's ethnic origin, language, cultural values and place of residence. The value conversion 1532 may define how expressions within a culture or population in regards to factors impacted by a disability such as pain, mobility expression, language terminology, or geographically related specific terminology or considerations. Further, additional question may be required to define specific conditions of an employee based upon the cultural group they are associated with to assess the impact of an injury.

The mapping may be used to convert graphics to different graphical formats, colours or expressions more appropriate to provide a better determination of a condition, injury or impact. For example graphic collection of data may be utilized rather than sentence based questions but result in the same normalized metric data to be collected. Similar the order in which data is collected may be modified based upon cultural preferences or how conditions are expressed. The mapping are used to modify based collection templates (1524) associated with the condition data. The presentation of the data collection to the employee if converted (1506) and the interface is presented (1508) to the employee to facilitate data collection (1510). The collected data can then be converted to normalized data scales (1512). For example if a graphic is utilized to determine pain level, the selected graphic can be mapped to a numeric value. The data can then be flatten to a standard format (1514) and processed against stored profiles as shown in FIG. 2 (208)

Each element in the embodiments of the present disclosure may be implemented as hardware, software/program, or any combination thereof. Software codes, either in its entirety or a part thereof, may be stored in a computer readable medium or memory (e.g., as a ROM, for example a non-volatile memory such as flash memory, CD ROM, DVD ROM, Blu-ray™, a semiconductor ROM, USB, or a magnetic recording medium, for example a hard disk). The program may be in the form of source code, object code, a code intermediate source and object code such as partially compiled form, or in any other form. Open Application Programming Interfaces have been developed for each screen so that sets of rules enables various programs to talk to it, regardless of the programming language they were written in. Thus, this software can be plugged into existing Enterprise Resource Management programs by talking to each other through a predefined URLs which can be accessed via HTTP Protocol. These URLs represent various resources. A resource represents a Business Object, such as an Appointment, a Customer, or an Admin User. Resources will be returned in a JSON format.

It would be appreciated by one of ordinary skill in the art that the system and components shown in FIGS. 1-15 may include components not shown in the drawings. For simplicity and clarity of the illustration, elements in the figures are not necessarily to scale, are only schematic and are non-limiting of the elements structures. It will be apparent to persons skilled in the art that a number of variations and modifications can be made without departing from the scope of the invention as defined in the claims. This application can be used in other industries where the ability to capture patient data for baseline measurement and ongoing maintenance is important to providing culturally appropriate healthcare, such as physician offices and hospital settings. This application can also be applied to payroll software or human resources management programs where having a person self-identify needs on a regular basis would be of benefit to improving workplace Diversity, Equity and Inclusion. This application can be linked to other healthcare devices where gathering input from multiple sources can be used to correlate past and predictive health behaviours.

1. A method of disability management comprising:
receiving at a management server identification of evaluation requirements for a disability condition associated with an employee of an associated case identifier;
generating a collection profile for the employee;
determining a cultural profile associated with the employee;
determine data collection requirements for associated disability condition;
converting data collection requirements based on cultural profile associated with the employee;
presenting a data collection interface to employee through a remote computing device;
receiving disability assessment responses through the data collection interface from the remote computing device;
converting received disability assessment responses to standard format from a determined translation mapping based upon the associated cultural profile;
processing collected data against a plurality of stored profiles;
generating a disability compensation profile;
determining expected outcomes associated with the disability compensation profile;
processing events associated with the disability compensation profile; and
generating one or more analytics from the processed events.

2. The method of claim 1 wherein the data collection requirements are associated with a base collection template, the base collection template defining one of more measures associated with the determined disability compensation profile.

3. The method of claim 1 wherein an employee language or cultural requirements is selected from a plurality of language or cultural definitions.

4. The method of claim 1 wherein the employee language or cultural requirements is defined by one or more of a language mapping, a condition expression terminology, rating scales, or graphic mappings.

5. The method of claim 1 further comprising determining if the generated compensation profile matches and existing profile.

6. The method of claim 5 further comprising generating a new profile category if an existing profile does not exist.

7. The method of claim 5 further comprising generating profile tasks and threshold based upon the existing profile.

8. The method of claim 7 further comprising determining an expected outcome for the disability compensation profile.

9. The method of claim 5 further comprising generating a recommended approval or denial for the case identifier is generated and presented for selection.

10. The method of claim 9 further comprising generating profile events when the case identifier is approved defining actions to be performed in relation to the employee claim process.

11. The method of claim 10 further comprising comparing collected case events with the determined existing profile and identifying when one or more thresholds are exceeded for the case identifier.

12. The method of claim 11 further comprising aggregating case identifiers into an aggregate event associated with a disability compensation profile.

13. A system for disability management, the system comprising:
a network interface coupled to a wide area network for communication with a computing device associated with an employee;
a processor coupled to the network interface for receiving and process data related to an employee injury claims; and
a computer readable memory containing instructions which are executed by the processor to perform the method of claim 1.

14. The system of claim 13 wherein the data collection requirements are associated with a base collection template, the base collection template defining one of more measures associated with the determined disability compensation profile.

15. The system of claim 13 wherein an employee language or cultural requirements is selected from a plurality of language or cultural definitions or is defined by one or more of a language mapping, a condition expression terminology, rating scales, or graphic mappings.

16. The system of claim 13 further comprising determining if the generated compensation profile matches and existing profile.

17. The system of claim 16 further comprising (i) generating a new profile category if an existing profile does not exist, (ii) generating profile tasks and threshold based upon the existing profile, or (iii) generating a recommended approval or denial for the case identifier is generated and presented for selection.

18. The system of claim 17 further comprising determining an expected outcome for the disability compensation profile.

19. The system of claim 17 further comprising generating profile events when the case identifier is approved defining actions to be performed in relation to the employee claim process.

20. The system of claim 19 further comprising comparing collected case events with the determined existing profile and identifying when one or more thresholds are exceeded for the case identifier, and aggregating case identifiers into an aggregate event associated with a disability compensation profile.
